(19) United States
(12) Patent Application Publication
(10) Pub. No.: US 2008/0081041 A1
(43) Pub. Date: Apr. 3, 2008

(54) Method of Using IL6 Antagonists with Mitoxantrone for Prostate Cancer

(76) Inventor: Jeffrey Nemeth, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/858,418

(22) Filed: Sep. 20, 2007

Related U.S. Application Data
(60) Provisional application No. 60/827,561, filed on Sep. 29, 2006.

(51) Int. Cl. A61K 39/395; A61P 29/00; A61P 35/00

(52) U.S. Cl. 424/145.1; 424/158.1

(57) ABSTRACT

The invention is directed to a method of treating a subject diagnosed with prostate cancer which comprises co-administering mitoxantrone in combination with an IL-6 antagonist.

### CLAIM TO PRIORITY

This application claims the benefit of U.S. Provisional Application Ser. No. 60/827,561, filed 29 Sep. 2006, the entire contents of which is incorporated herein by reference in its entirety.

### BACKGROUND OF THE INVENTION

1. Field of the Invention

The present invention relates to methods for treating cancer in a subject by administering to a subject an effective amount of an immunosuppressive anthracendione and an effective amount of an interleukin-6 antagonist. The present invention relates to the use of an interleukin-6 antagonist to enhance the response of treatment of a subject being treated for diseases, such as cancer, with an immunosuppressive anthracenedione such as mitoxantrone. The present invention particularly relates to antibodies, including specified portions or variants, specific for Interleukin-6 (IL-6 also known as Interferon β2)) protein.

2. Background

### Cytokine IL-6

IL-6 (interleukin 6) is a 22-27 kDa secreted glycoprotein formerly known as monocyte-derived human B-cell growth factor, B-cell stimulatory factor 2, BSF-2, interferon beta-2, and hybridoma growth factor, which has growth stimulatory and proinflammatory activities (Hirano et al. Nature 324: 73-76, 1986).

IL-6 belongs to the granulocyte colony-stimulating factor (G-CSF) and myelomonocytic growth factor (MGF) family which includes leukemia inhibitory factor (LIF), oncostatin M (OSM), ciliary neurotropic factor (CNTF), cardiotropin-1 (CT-1), IL-1, and IL-11. IL-6 is produced by an array of cell types, most notably antigen presenting cells, T cells and B cells. IL-6-type cytokines all act via receptor complexes containing a common signal transducing protein, gp130 (formerly IL-6Rbeta). However, whereas IL-6, IL-11, CT-1, and CNTF bind first to specific receptor proteins which subsequently associate with pg130, LIF and OSM bind directly to a complex of LIF-R and gp130. The specific IL-6 receptor (IL-6R or IL-6alpha, gp80, or CD126) exists in either membrane bound or soluble forms (sIL-6R, a 55 kD form), which are both capable of activating gp130.

Several agents are known to induce the expression of IL-6 such as IL-1, IL-2, TNFa, IL-4, IFNa, oncostatin and LPS. IL-6 is involved in diverse activities such as B and T cell activation, hematopoiesis, osteoclast activity, keratinocyte growth, acute phase protein synthesis, neuronal growth and hepatocyte activation (Hirano et al. Int. Rev. Immunol; 16(3-4):249-84, 1998). Although IL-6 is involved in many pathways, IL-6 knockout mice have a normal phenotype, they are viable and fertile, and show slightly decreased number of T cells and decreased acute phase protein response to tissue injury (Kopf M et al. Nature: 368:339-42, 1994). In contrast, transgenic mice that over-express cerebral IL-6 develop neurologic disease such as neurodegeneration, astrocytosis, cerebral angiogenesis, and these mice do not develop a blood brain barrier (Campbell et al. PNAS 90: 10061-10065, 1993).

Increased levels of IL6 has been associated with ligand-independent activation of androgen receptor in prostate cancer cells and therefore be a factor in prostate cancer cell growth and metastasis. Prostatic tumor characteristically metastasizes to bone, lymph node and liver, where IL6 is present (Siegall et al., 1990; Siegsmund et al., 1994). An inverse correlation between circulating androgens and IL6 has been noted in normal men and prostate cancer. Androgens decrease with age while circulating IL6 increases. Patients with advanced prostate cancer have elevated systemic serum IL6, which is correlated with the tumor burden (Akimoto et al., 1998; Adler et al., 1999).

Experimental results from a number of in vitro and in vivo models of various human cancers have demonstrated that IL-6 is a therapeutic target for inhibition. IL-6 can induce proliferation, differentiation and survival of tumor cells, promote apoptosis (Jee et al. Oncogene 20: 198-208, 2001), and induce resistance to chemotherapy (Conze et al. Cancer Res 61: 8851-8858, 2001).

### Anthracenedione Chemotherapeutic Agents

Mitoxantrone (NOVANTRONE®) is a synthetic antineoplastic anthracenedione for intravenous use of the formula 1,4-dihydroxy-5,8-bis[[2-[(2-hydroxyethyl)amino]ethyl]amino]-9,10-anthracenedione dihydrochloride (CAS Reg. No. 65271-80-9). It intercalates into deoxyribonucleic acid (DNA) through hydrogen bonding and causes crosslinks and strand breaks. Mitoxantrone also interferes with ribonucleic acid (RNA) and is a potent inhibitor of topoisomerase II, an enzyme responsible for uncoiling and repairing damaged DNA. Mitoxantrone is cytocidal to both proliferating and nonproliferating cultured human cells. NOVANTRONE® has been shown in vitro to inhibit B cell, T cell, and macrophage proliferation and impair antigen presentation, as well as the secretion of interferon gamma, TNF(alpha), and IL-2. Mitoxantrone and other 9,10-anthracenediones have immunosuppressive activity in vitro and/or in vivo (Fidler, J. et al. 1986 J Immunol 137:727-732; Fidler, J. et al. 1986 J Immunol 136: 2747-2754; Wang, B. S. et al. 1987 Int J Immunopharmac. 9:733-9). In 2000, the FDA approved mitoxantrone for worsening relapsing-remitting multiple sclerosis as, in addition to other activities, it inhibits macrophage-mediated myelin degradation (Fox, E. J. 2004 Neurology 63(Suppl 6): S15-S18).

### Prostate Cancer

Prostate adenocarcinoma is the most common malignancy in men one of the most important health problems in industrialized countries. It is the second leading cause of cancer-related death in the United States. Therapeutic options are different according to the stage of the disease at the diagnosis. Patients with localized disease may be treated with surgery or radiation, whereas the treatment for patients with a metastatic disease is purely palliative. Hormonal treatment represents the standard therapy for stage 1V prostate cancer, but patients ultimately become unresponsive to androgen ablation and are classified as hormone-refractory prostate cancer (HRPC) patients. Initial treatment of metastatic disease by orchiectomy or by drugs that ablate androgens relieves symptoms in approximately 75% of cases but all eventually progress to hormone resistant disease. Median survival of HRPC patients is approximately 9 to 12 months.

Conventional options for HRPC patients include secondary hormone therapy, radiotherapy and cytotoxic chemotherapy. A combination of mitoxantrone and prednisone is approved for the palliation of symptomatic patients with hormone refractory prostate cancer. New drugs and new combinations have shown increased activity especially those including the antineoplastic agents estramustine and taxanes. For example, the semisynthetic taxane docetaxel given with estramustine reported a median survival of 20 months in some patients involved in clinical studies.

Therefore, new approaches which could provide a survival benefit in the treatment of hormone-refractory prostate cancer are needed. The advantageous effects of combining biologic drugs such as cytokine inhibitors, specifically IL6 antagonists, with an immunosuppressive anthracenedione drugs has heretofore not been demonstrated.

### SUMMARY OF THE INVENTION

The present invention relates to methods for treating disease in a subject by administering to a subject an effective amount of an immunosuppressive 9,10-anthracenedione and an effective amount of an interleukin-6 antagonist. The method of the invention comprises administration of an anti-IL6 antagonist sequentially, serially, or concurrently with mitoxantrone or related 9,10-anthracenedione. In one embodiment, the IL6 antagonist is a high affinity anti-IL6 antibody. Subjects suffering from a disease amenable to the method of the invention include those subjects diagnosed with various forms of cancer, a neuroinflammatory disease such as multiple sclerosis, and other autoimmune disease. In one embodiment, the disease is prostate cancer. In a specific embodiment, the subject is diagnosed with prostate cancer and said subject has undergone administration of androgen ablation therapy.

The present invention further provides a method for predicting the utility of a combination of at least one IL-6 antagonist and at least one immunosuppressive 9,10-anthracenedione using animal models of cancer.

### BRIEF DESCRIPTION OF THE DRAWINGS

FIG. 1 is a graph showing the relationship of concentration of mitoxantrone to cell proliferation by DU145 androgen-independent prostatic adenocarcinoma cells in culture at three different times of incubation: 24, 48 and 72 hours.

FIG. 2 is a graph plotting the median tumor volumes in four groups of nude mice treated with PBS, CNTO328, Mitoxantrone or the combination of CNTO328 and mitoxantrone over a 56 day experiment.

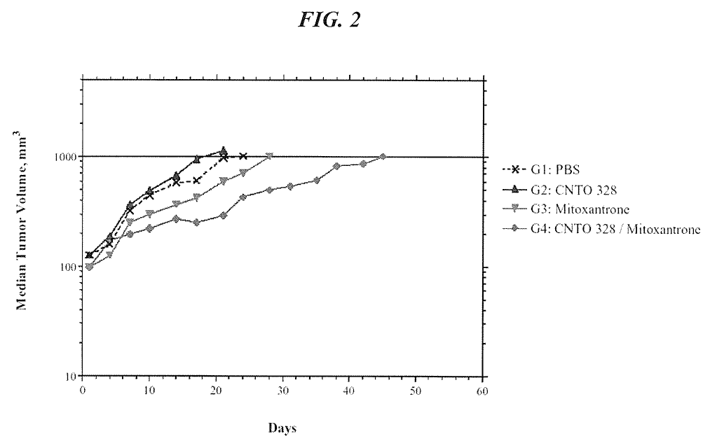

### DETAILED DESCRIPTION OF THE INVENTION

### Abbreviations

AE adverse event; ECG electrocardiogram, Ig immunoglobulin, IgG immunoglobulin G, IL interleukin, IL6 interleukin-6, IL-6R interleukin-6 receptor, sIL-6R soluble interleukin-6 receptor, Mab monoclonal antibody, M or MX mitoxantrone, STAT signal transduction activation,

### Definitions

By “androgen ablation therapy” is meant any procedure or course of therapy intended to reduce or eliminate the level of androgen receptor ligands in the body of the patient. As the testes are the responsible 90% of androgen production in men, androgen can be reduced by biological (orchiectomy) or chemical castration. Additive or ablative endocrine therapy can influence the course of some cancers. Endocrine therapy is not curative; it is only palliative. Orchiectomy has significant palliative value in metastatic prostate cancer, commonly prolonging survival 3 to 5 yr. Its efficacy is based on the testosterone-dependent population of prostate cancer cells. Other cancers with hormone receptors on their cells (eg, breast, endometrium, ovary) can often be palliated by hormone ablative therapy. Estrogen effectively palliates prostate cancer but increases the risk of heart disease. Another treatment approach is with gonadotropin secretory inhibitors. Leuprolide, a synthetic analog of gonadotropin-releasing hormone, inhibits gonadotropin secretion and resultant gonadal androgen production and is as effective for the palliation of prostate cancer as is orchiectomy. Even more complete androgen blockage can be achieved by adding an oral antiandrogen (e.g. flutamide or bicalutamide), which limits androgen binding to its receptor and increases disease-free survival time over leuprolide or orchiectomy alone.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibody fragments” comprise a portion of a full length antibody, generally the antigen binding or variable domain thereof. Examples of antibody fragments include Fab, Fab′, F(ab′)2, and Fv fragments; diabodies; linear antibodies; single-chain antibody molecules; and multispecific antibodies formed from antibody fragments.

“Chimeric antibodies” are those antibodies that retain distinct domains, usually the variable domain, from one species and the remainder from another species; e.g. mouse-human chimeras.

The term “human antibody”, as used herein, is intended to include antibodies having variable and constant regions derived from or closely matching human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo such as during the recombination of V, D, and J segments of the human heavy chain). Thus, as used herein, the term “human antibody” refers to an antibody in which substantially every part of the protein (e.g., CDR, framework, CL, C_{H} domains (e.g., C_{H}1, C_{H}2, C_{H}3), hinge, (V_{L}, V_{H})) is substantially similar to those encoded by human germline antibody genes. Human antibodies have been classified into groupings based on their amino acid sequence similarities, see e.g. http://people.cryst.bbk.ac.uk/˜ubcg07s/. Thus, using a sequence similarity search, an antibody with similar linear sequence can be chosen as a template to select or create human or humanized antibodies.

As used herein, the term “high affinity” for an antibody refers to an antibody having a K_{D} Of 10⁻⁸ M or less, more preferably 10⁻⁹ M or less and even more preferably 10⁻¹⁰ M or less. The term “Kdis” or “K_{D},” or “Kd’ as used herein, is intended to refer to the dissociation rate of a particular antibody-antigen interaction. The “K_{D}”, is the ratio of the rate of dissociation (k₂), also called the “off-rate (k_{off})”, to the rate of association rate (k₁) or “on-rate (kₒₙ)”. Thus, K_{D} equals k2/k1 or k_{off}/kₒₙ and is expressed as a molar concentration (M). It follows that the smaller K_{D}, the stronger the binding. So a K_{D} of 10⁻⁶M (or 1 μM) indicates weak binding compared to 10⁻⁹ M (or 1 nM).

As used herein, “an immunosuppressive 9,10-anthracenedione” is defined as a 9,10-anthracenedione which can inhibit the proliferation of B-lymphocytes (B-cells), T-lymphocytes (T-cells), or macrophages or suppress their biological activity. Assays for determining 9,10-anthracenedione immunosuppressive activity are taught in e.g. Fidler, J. et al. 1986 J Immunol 137:727-732; Fidler, J. et al. 1986 J Immunol 136: 2747-2754; Wang, B. S. et al. Int J Immunopharmac. 9:733-9, 1987.

The term “apoptosis” or “undergoes apoptosis” refers to a specific pattern of cell death characterized by the internal degradation of intracellular structures and major components, principally the nucleus and chromosomal DNA, prior to disruption or lysis of the plasma membrane and is also referred to as “programmed cell death”. Thus, tumor cells, cells of the immune system, or other cells may be destroyed by an agent which causes the cell to initiate the program leading to death or “apoptosis”.

As used herein the term “synergistic” defines an measured response, such as tumor growth inhibition or cell death resulting from the biological action due to the presence of more than one agent, which is quantitatively greater, larger in magnitude, than the additive measured response resulting in the absence of each individual agent at the same effective concentration. For example, if the endpoint of a test is a direct or indirect measurement of the surviving fraction of a population of cells, the surviving fraction or percentage of cells surviving as a result of treatment of the cells with one agent would be multiplied by the surviving fraction of cells resulting from treatment with a second agent to give the expected surviving fraction due to the independent effects of both agents. If the surviving fraction of cells treated with both agents is smaller than the product of fractions for each individual agent alone, the effect can be termed synergistic. In another example where the effect of an agent is measured by, e.g. tumor growth, the effects of individual agents are quantitated by the measurement of tumor size, tumor growth rate (time to reach a predetermined size), or the sequelae of the tumor growth as in survival of the host. The “synergistic” effect can be further qualified as quantitatively greater through the use of the appropriate statistical analysis where repeated measurements are made. Thus, where repeated measures are used the analysis of variance can be used to exclude to possibility that the synergistic effect is due to random chance.

As used herein, the term “resistant” or “refractive” to a therapeutic agent when referring to a cancer cell means that the cell has achieved resistance to the effects of the agent normally caused by exposure to a environmental level or concentration of that agent with impairs or inhibits proliferation, or is inhibited to a very low degree, as a result of contact with the level of therapeutic agent when compared to when normal or nonresistant cells are brought in contact with the same level or concentration of the therapeutic agent. The quality of being resistant to a therapeutic agent is a highly variable one, with different cancer cells exhibiting different levels of “resistance” to a given therapeutic agent under different conditions.

### Prostate Cancer Diagnoses

Adenocarcinoma of the prostate is the most common malignancy in men over 50 years of age. Sarcoma of the prostate is rare, occurring primarily in children. Undifferentiated prostate cancer, squamous cell carcinoma, and ductal transitional carcinoma also occur and respond poorly to the usual measures of control. Hormonal influences undoubtedly play a role in the etiology of adenocarcinoma but almost certainly no role in sarcoma, undifferentiated cancer, squamous cell carcinoma, or ductal transitional cell carcinoma.

As used herein, the term “advanced prostate cancer” is meant clinical disease which is palpable or visible or confirmed in specimens by any means, such as by histology.

Prostate cancer is usually glandular and similar to the histologic configuration of normal prostate. Small cell proliferation and large nucleoli are characteristic. Although most cancers arise near the capsule in the peripheral zone, the disease is generally multifocal, and tumors are often present throughout the gland. Spread may occur by local extension through defects in the capsule where the neurovascular structures and the ejaculatory ducts enter the gland or in the region of the bladder neck. Local invasion can progress to involve the seminal vesicles or the bladder or to invade the levator muscles. Rarely does a tumor invade the rectal wall. Tumors of the apex are prone to early extracapsular extension (ECE) due to a weakness of the capsule in this location. Systemic spread can occur via the lymphatics to involve the obturator, hypogastric, presacral, and external iliac nodes or hematogenously to involve bone, lung, or liver. Prostate cancers in particular have a predilection for bone, in part owing to a unique bidirectional interaction between tumor cells and the surrounding stroma.

Prostate cancer generally is slowly progressive and may cause no symptoms. In late disease, symptoms of bladder outlet obstruction, ureteral obstruction, and hematuria may appear. Metastases to the pelvis, ribs, and vertebral bodies may cause bone pain. Locally advanced prostate cancer may exhibit extension of induration to the seminal vesicles and fixation of the gland laterally.

Prostate cancer should be suspected on the basis of abnormal digital rectal findings, hypoechoic lesions on transrectal ultrasound (TRUS), or elevated levels of serum prostate-specific antigen (PSA). PSA (NCBI Accession No. NP_{—}001639) is a kallikrein-like serine protease that causes liquefaction of seminal coagulum. Kallikreins are a subgroup of serine proteases having diverse physiological functions. This gene is one of the fifteen kallikrein subfamily members located in a cluster on chromosome 19. Alternate splicing of this gene generates several transcript variants encoding different isoforms. PSA is produced by both nonmalignant and malignant epithelial cells. PSA is prostate specific, not prostate cancer specific, and increases may occur from prostatitis, nonmalignant enlargement of the gland (BPH), prostate cancer, and prostate biopsies. It circulates in the blood as an inactive complex with the protease inhibitors-1-antichymotrypsin and 2-macroglobulin and has an estimated half-life in the serum of 2 to 3 days. Levels should be undetectable if the prostate has been removed. PSA immunostaining is used to establish a prostate cancer diagnosis.

Elevated PSA alone with or without positive finding in a digital rectal exam (DRE) is insufficient to diagnose carcinoma and histologic confirmation is required, most commonly by TRUS-guided transrectal needle biopsy, which can be done in the clinic without anesthesia. Involvement of perineural lymphatics, if present, is diagnostic. Carcinoma is diagnosed incidentally when malignant changes are found in the tissue removed during surgery for suspected benign prostatic enlargement. Prostate cancer frequently produces osteoblastic bony metastases. Detection on bone scan or x-ray in the presence of a stony hard prostate is usually diagnostic.

TRUS may provide information for staging, particularly relative to capsular penetration and seminal vesicle invasion. Elevated serum acid phosphatase on Roy test (an enzymatic method) correlates well with the presence of metastases, particularly in lymph nodes. This enzyme may also be elevated in benign prostatic hyperplasia (slight elevation after vigorous prostatic massage), multiple myeloma, Gaucher's disease, and hemolytic anemia.

PSA is the most sensitive marker for monitoring cancer progression and response to therapy. However, because serum PSA is moderately elevated in 30 to 50% of patients with benign prostatic hyperplasia (depending on prostate size and degree of obstruction) and in 25 to 92% of those with prostate cancer (depending on tumor volume), its role in early detection and staging is still being evaluated. Significantly elevated PSA levels suggest extracapsular extension of tumor or metastases. Assays that determine the proportion of free vs. bound PSA may also be used.

### Staging

Prostate cancers are staged using the TNM (tumor, node, metastasis) classification developed by the American Joint Committee on Cancer and the International Union Against Cancer, first published in 1992 (F F Schroder et al: TNM classification of prostate cancer. Prostate (Suppl) 4:129, 1992; and American Joint Committee on Cancer, 1992) and revised in 1997 and again in 2002 (Table 1). With the TNM system, designations for the primary tumor, regional nodes, and distant metastases are noted separately. A distinct category, T1c, is used to describe cancers that are neither palpable nor visible but were detected by a biopsy performed because of an abnormal PSA or another reason. Cancers that are not palpable but are visible by an imaging study, such as transrectal ultrasound (TRUS) or magnetic resonance imaging (MRI), are classified appropriately along with palpable cancers in the T2 to 4 categories. The 2002 system, like the 1992 version, established three T2 categories—a, b, and c.

TABLE 1

| | |
|---|---|
| T1 | Clinically inapparent, not palpable or visible by imaging |
| T1a | Incidental histologic finding, ≦5% of resected tissue |
| T1b | Incidental histologic finding, >5% of resected tissue |
| T1c | Tumor identified by needle biopsy, for any reason |
| | (e.g., elevated PSA) |
| T2 | Palpable or visible tumor, confined within the prostate |
| T2a | ≦½ one lobe |
| T2b | One lobe |
| T2c | Both lobes |
| T3 | Tumor extends through the capsule |
| T3a | ECE, unilateral or bilateral |
| T3b | Bilateral ECE Seminal vesicle involvement |
| T3c | Seminal vesicle involvement |
| T4 | Tumor is fixed or invades adjacent structures |
| T4a | Invades bladder neck, external sphincter or rectum |
| T4b | Invades levator muscles or fixed to pelvic sidewalls |

The major cause of death from prostate cancer is progressive castration-resistant disease, that is, a tumor that continues to grow despite castrate levels of testosterone, also called “hormone resistant” prostate cancer (HRPC). As prostate cancers evolve to HRPC, PSA synthesis resumes. The current view is that prostatic cancers at the time of diagnosis are composed of cells with three distinct cellular phenotypes: androgen-dependent, androgen-sensitive, and androgen-independent cells. Androgen-dependent cancer cells continuously require a critical level of androgenic stimulation for maintenance and growth (i.e., without adequate androgenic stimulation, these cells die) and, in this regard, are very similar to the androgen-dependent normeoplastic cells of the normal prostate. The growth of androgen-sensitive cancer cells slows when androgens are withdrawn. In contrast, the growth of androgen-independent cells does not change after androgen deprivation.

The androgen receptor (dihydrotestosterone receptor, AR, NCBI Accession No. P10275) is a member of a super-family of ligand-dependent transcription factors. The AR gene is located on chromosome Xq11-13 and spans eight exons, whereas the AR protein has three functional domains: a large, highly variable amino-terminal domain (NTD) encoded entirely by exon 1 that contains two regions with strong transactivation functions, AF-1 and AF-5; a DNA-binding domain encoded by exons 2 and 3; and a carboxy-terminal ligand-binding domain encoded by exons 4 through 8 that contains a highly conserved ligand-dependent transactivation function (AF-2). Binding of high-affinity ligands induces conformational changes that lead to the recruitment of coregulator proteins: coactivators that enhance or corepressors that repress AR function.

Alterations in AR signaling that have been identified in human prostate cancer include alterations in steroid metabolism, an increase in the level of the protein, changes in coregulator profiles, and androgen-independent activation. Changes in AR occur as the disease progresses from a clinically localized lesion in a noncastrate environment to a castrate metastatic lesion. All of these mechanisms are consistent with continued signaling through the receptor in castration-resistant lesions.

In addition to steroid hormones, growth factors, such as keratinocyte growth factor, IGF-1, and EGF; HER2; and cytokines, such as interleukin-6 (IL-6), can be shown to cause AR signaling independent of ligand. AR activity contributes to progression in castration-resistant disease.

Finasteride (PROSCAR) is a synthetic 4-azasteroid compound which is a specific inhibitor of steroid Type II 5(alpha)-reductase, an intracellular enzyme that converts the androgen testosterone into 5(alpha)-dihydrotestosterone (DHT). The development and enlargement of the prostate gland is dependent on the potent androgen, 5(alpha)-dihydrotestosterone (DHT). Type II 5(alpha)-reductase metabolizes testosterone to DHT in the prostate gland, liver and skin. DHT induces androgenic effects by binding to androgen receptors in the cell nuclei of these organs. Finasteride is used to treat benign prostatic hypertrophy but has not shown a clinical benefit in the treatment of prostate cancer.

Inhibitors of apoptosis are also implicated in the acquisition of the castration-resistant phenotype. Blocking cell death pathways that are normally induced by androgen ablation allows cells to survive. BCL-2, which inhibits the death of cancer cells without affecting their rate of proliferation, is essentially undetectable in most noncastrate lesions but is highly expressed in castration-resistant disease. Similarly, survivin, a member of the class of proteins called inhibitors of apoptosis, is highly expressed in benign and malignant prostate neuroendocrine cells. Survivin functions to inhibit effector caspases.

The potential use of CNTO 328, an anti-IL-6 monoclonal antibody, in treating prostate cancer was first demonstrated in a xenograft model of human hormone-refractory prostate tumor in mice. In this model, anti-IL-6 mAb, the murine CNTO 328, regressed established tumors and induced tumor cell apoptosis (Smith and Keller, 2001 Prostate. 48(1):47-53). In a recent study, CNTO 328 monotherapy has also been shown to block conversion to androgen independent growth, induce tumor apoptosis, and prolong survival of human prostate tumor-bearing mice (Wallner et al, 2006).

### Bone Metastasis

Prostate cancer cells that escape the capsule and gain access to the circulation, in stage T3 and higher, are bone seeking and driven, in part, by a chemoattractant gradient of marrow- and stromal-derived growth factors. Once established, tumor cells and marrow-derived cells develop a bidirectional interaction that protects the epithelial cells and promotes tumor cell survival and proliferation.

Radiographically, metastatic prostate cancers are primarily osteoblastic or bone-building. Osteoclast (bone-degrading cells) stimulation and activation continues, however, as evidenced by increased levels of markers of bone turnover. Thus, the normal bone remodeling process is shifted in favor of bone growth. It is hypothesized that the resorptive process itself, under the direction of osteoclasts, promotes the release of factors that amplify the metastatic and invasive process. The proteolytic action of PSA results in the activation of functional signaling molecules adjacent to tumor that further contribute to tumor cell growth and proliferation. For example, PSA cleavage of IGF, from its binding protein (IGFBP3), increases the local levels of a functional prostate cancer mitogen that is normally inactive as a bound complex. PSA can also activate parathyroid hormone-related protein (PTH), which inhibits osteoblast apoptosis.

### Prognosis and Treatment

Long-term local control or cure depends on factors such as grade, stage, and pretreatment PSA level. For patients with low-grade, organ-confined tumors, survival is virtually identical to that for age-matched controls without prostate cancer.

Patients may elect to undergo definitive therapy with radical prostatectomy or radiotherapy. Radical prostatectomy is accompanied by the risk of urinary incontinence but erectile potency can be maintained (if at least one neurovascular bundle can be spared). Radiotherapy may offer comparable results, especially in patients with low pretreatment PSA levels. Standard external beam radiotherapy generally delivers 70 Gy (7000 rad) in 7 wk. Conformal three-dimensional techniques safely deliver doses approaching 80 Gy (8000 rad), or interstitial irradiation (seed implants) can be used.

An asymptomatic patient with a locally advanced tumor or metastases may benefit from hormonal therapy with or without adjuvant radiotherapy. Hormonal therapy rarely uses exogenous estrogens, which pose a risk of cardiovascular and thromboembolic complications. Bilateral orchiectomy or medical castration with luteinizing hormone-releasing hormone agonists decreases serum testosterone equivalently. Some patients may benefit from the addition of oral antiandrogens: flutamide, bicalutamide, or nilutamide; for total androgen blockade. Local radiotherapy is usually palliative in patients with symptomatic bone metastases.

Medical therapies can be divided into those that lower testosterone levels, e.g., gonadotropin-releasing hormone (GnRH) agonists and antagonists, estrogens and progestational agents, and the antiandrogens that bind to the androgen receptor but do not signal. Ketoconazole inhibits adrenal androgen synthesis and is used after first-line castration is no longer effective. In this setting, the adrenal glands may contribute up to 40% of the active androgens in the prostate.

At the time of this writing, there is no standard therapy for hormone refractory prostate cancer; multiple regimens investigating biologic agents with and without cytotoxic chemotherapeutic agents are being investigated and compared to corticosteroids alone. No chemotherapy regimen has been proven to prolong life in these patients. Drugs directed at the tumor cell cytoskeleton such estramustine (EMCYT) and a taxane such as paclitaxel or docetaxel (TAXOTERE) can induce responses in 50% using measurable disease regression as the endpoint. Seventy percent show a >50% decline in PSA from baseline. Docetaxel, estramustine, and combinations with vinorelbine (NAVELBINE) have also been used.

Mitoxantrone has been found to offer palliative management of patients with advanced prostate cancer, especially androgen-independent and hormone refractory disease which was established in two randomized trials of mitoxantrone and prednisone vs. prednisone alone. In both studies, mitoxantrone-treated patients had a greater reduction in pain, used fewer narcotics, were more mobile, and had less fatigue. No survival benefit was shown.

Metastases to the bone may be managed with bisphosphonate drugs such as clodronate or zoledronate or other palliative measures such as irradiation. Two bone-seeking radioisotopes, 89Sr (metastron) and ¹⁵³Sm-EDTMP (quadramet), are approved for palliation of pain although they have no effect on PSA or on survival. Addition of zoledronate to “standard therapy” in patients with castration-resistant disease resulted in fewer skeletal events relative to placebo-treated patients. Patients randomly assigned to a combination of 89Sr and doxorubicin after inducation chemotherapy had fewer skeletal events and longer survival than patients treated with doxorubicin alone. Confirmatory studies are ongoing.

### IL6 Antagonists of the Invention

The IL-6 antagonist used in the present invention may be of any origin provided it blocks signal transmission by IL-6, and inhibits the biological activity of IL-6. Examples of IL-6 antagonists include IL-6 antibody, IL-6R antibody, gp 130 antibody, IL-6 mutant, IL-6R antisense oligonucleotide, and partial peptides of IL-6 or IL-6R. An example of the IL-6 mutant used in the present invention is disclosed in Brakenhoff, et al., J. Biol. Chem., 269, 86-93, 1994 or Savino, et al., EMBO J., 13, 1357-1367, 1994. The IL-6 mutant polypeptide or fragment thereof does not possess the signal transmission effects of IL-6 but retains the binding activity with IL-6R, and is produced by introducing a mutation in the form of a substitution, deletion or insertion into the amino acid sequence of IL6. While there are no limitations on the animal species used, it is preferable to use an IL6 of human origin. Similarly, any IL-6 partial peptides or IL-6R partial peptides used in the present invention provided they prevent IL6 or IL6R (gp80) or gp130 from affecting signal transduction and thereby prevent IL-6 associated biological activity (U.S. Pat. No. 5,210,075; EP617126 for details regarding IL-6 partial peptides and IL-6R partial peptides). In yet another embodiment, oligonucleotides capable of IL6 or IL6R RNA silencing or antisense mechanisms can be used in the method of the present invention (JP5-300338 for details regarding IL-6R antisense oligonucleotide).

### Antibodies of the Invention

Antibodies useful in the present invention include isolated chimeric, humanized and/or CDR-grafted, or human antibodies, having at least one antigen-binding region which are capable of inhibiting the biological functions of IL6. Examples of antibodies of the invention include IL-6 binding antibody, IL-6R (gp80) binding antibody, gp130-binding antibody. Examples of IL-6R antibodies with suitable antigen binding regions include PM-1 antibody (Hirata, et al., J. Immunol., 143, 2900-2906, 1989), and AUK12-20, AUK64-7 or AUK146-15 antibody (WO92-19759). In another embodiment, the anti-IL6R antibody is the reshaped antibody known as MRA disclosed in U.S. Pat. Nos. 5,888,510 and 6,121,423.

In one embodiment the antigen binding region is derived from the high affinity CLB-8 anti-IL-6 antibody. An exemplary antibody of the invention derived from CLB-6 is CNTO328 as described in applicants co-pending application U.S. Ser. No. 10/280,716 the contents of which are incorporated herein by reference. In an alternate embodiment, the antibody is a human antibody which binds IL6 with high affinity such as is described in applicants co-pending U.S. provisional patent application Ser. No. 60/677,319. The antibody of the invention specifically neutralizes human IL-6 with high affinity.

An anti-IL-6 antibody which may be used in the method according to the present invention includes any protein or peptide molecule that comprises at least one complementarity determining region (CDR) of a heavy or light chain or a ligand binding portion thereof, derived from the murine CLB-8 monoclonal antibody, in combination with a heavy chain or light chain constant region, a framework region, or any portion thereof, that can be incorporated into an antibody of the present invention. In one embodiment the invention is directed to an anti-IL-6 chimeric antibody comprising two light chains and two heavy chains, each of the chains comprising at least part of a human constant region and at least part of a variable region (v) derived from the murine c-CLB8 monoclonal antibody having specificity to human IL-6, said antibody binding with high affinity to an inhibiting and/or neutralizing epitope of human IL-6, such as the antibody cCLB-8. The invention also includes fragments or a derivative of such an antibody, such as one or more portions of the antibody chain, such as the heavy chain constant, joining, diversity or variable regions, or the light chain constant, joining or variable regions.

Preferred antibodies of the present invention include those chimeric, humanized and/or CDR grafted, or human antibodies that will competitively inhibit in vivo binding to human IL-6 of anti-IL-6 murine CLB-8, chimeric anti-IL-6 CLB-8, or an antibody having substantially the same binding characteristics, as well as fragments and regions thereof.

The antibody of the invention preferably binds anti-IL6 or anti-IL6R with an affinity (K_{d}) of at least 10⁻⁹ M, preferably at least 10⁻¹⁰ M, and/or substantially neutralize at least one activity of at least one IL-6 protein. In a preferred embodiment, the antibody binds IL-6 with an affinity (K_{d}) of at least 1×10⁻¹¹ M, preferably 5×10⁻¹¹ neutralizes human IL-6. Preferably, the antibody does not bind other IL-6 superfamily members and blocks trans-signaling of GP 130.

### Immunosuppressant Anthroquinones

Mitoxantrone and a structurally related molecule with similar properties ametantrone (AQ, Cas Reg. No. 64862-96-0) were described in Zee-Cheng, R. et al., 1978. J. Med. Chem., 21: 291-4. Based on the desirable characterisitics of these molecules as antineoplastic agents, Krapcho et al. (1985 J. Med. Chem. 28: 1124-1126) developed a novel class of anthracene-9,10 diones characterized by the introduction of a nitrogen functionality in the nucleus and by the lack of the two hydroxy groups (likely involved in the cardiotoxicity) as a result of the replacement of the 5,8-dihydroxyphenyl ring of mitoxantrone by a pyridine ring. One of them (6,9-bis[(2 aminoethyl)amino]benz[g]isoquinoline-5,10 dione) dimaleate salt (BBR2778, pixantrone), exhibits antitumor activity comparable to mitoxantrone but with reduced toxicity to cardiac tissue after single- and multiple-dose treatment in animals. Based on the immunosuppressive properties of pixantrone, it is being tested for treatment of patients with multiple sclerosis. The structures of 9-10-anthracenediones of the invention are shown below as formula I:

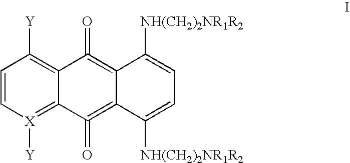

### X=C or N

### Y=H or OH, with the provisio that if X═N, Y═H

### R1=H, CH₃, CH₂CH₃

### R2=H, CH₃, CH₂CH₃, (CH₂)₂OH

When X═C, Y═H, R₁═H; R₂═(CH2)₂OH; the compound is ametantrone; 1,4-Bis[(2-(2-hydroxyethylamino)ethyl)amino]-anthraquinone; 1,4-Bis[(2-(2-hydroxyethylamino)ethyl)amino]-9,10-anthracenedione; HAQ; CAS Reg. No. 64862-96-0.

When X═C, Y═OH, R₁═H; R₂═H; the compound is known as AEAD; 1,4-bis[(2-aminoethyl)amino]-5,8-dihydroxy-9,10-anthracenedione; 1,4-Bis[(2-aminoethyl)amino]-5,8-dihydroxyanthraquinone; CAS Reg. No. 96555-65-6.

When X═C, Y═OH, R₁═H; R₂=(CH2)₂OH; the compound is Mitoxantrone; 1,4-Dihydroxy-5,8-bis(2-[(2-hydroxyethyl)aminoethyl]amino)-9,10-anthracenedione; 1,4-Bis[(2-(2-hydroxyethylamino)ethyl)amino]-5,8-dihydroxyanthraquinone; 1,4-Dihydroxy-5,8-bis-[[2-[(2-hydroxyethyl)amino]ethyl]amino]anthraquinone; 1,4-Dihydroxy-5,8-bis[[2-[(2-hydroxyethyl)amino]ethyl]amino]-9,10-anthracenedione; DHAD; DHAQ; Dihydroxyanthraquinone; Mitoxanthrone; Mitoxantrone; Mitozantrone; NSC 279836; Novantron; Novantrone; Ralenova; CAS Reg. No. 65271-80-9.

When X═N and Y═H, the compounds can be described as aza-anthracene-9-10-diones. An exemplary compound of this type is when X═N, Y═H, and R₁═R₂═H; pixantrone; 6,9-bis[(2-amino)ethyl]amino)-benzo[g]isoquinoline-5,10-dione; BBR2778. The complete description of the compound BBR 2778 is reported in U.S. Pat. No. 5,587,382, U.S. Pat. No. 5,717,099, U.S. Pat. No. 5,506,232, U.S. Pat. No. 5,616,709 and in J. Med. Chem., 1994, Vol. 37, 828-837.

The 9,10-anthracenediones of the invention are functionally defined as having immunosuppressive activity in one or more in vitro or in vivo models. The most specific immune property of mitoxantrone is a dramatic drop in splenic and circulating B cells (Fidler, et al. 1986 J Immunol 136: 2747-54). Thus, mitoxantrone produces a marked suppressive effect on most B cells functions: antigen presentation, antibody-dependent demyelination and complement mediated myelinolysis. Importantly for the treatment of multiple sclerosis, mitoxantrone inhibits activation of CD4 cells by macrophages and their demyelinating activity. Mitoxantrone depresses helper CD4 and CD8 functions, while specific suppressor activity is spared. In addition, mitoxantrone induces the proliferation of nonspecific suppressor cells. Mitoxantrone thus broadly suppresses cells involved in autoimmune mechanisms.

Experimental allergic encephalomyelitis (EAE), an animal model of neurodegenerative disease with pathological similarity to multiple sclerosis in humans, can be used to demonstrate the immunosuppressive and myelin sparing effects of mitoxantrone and other anthracene-9,10 diones.

### Acute EAE Model

EAE can be actively induced in inbred rats by subcutaneous inoculation of guinea pig myelin basic protein (gpMBP, purified from spinal cords with the method of Deibler, Deibler et al., 1972) into both hind limb footpads of 50 μg in 100 μl complete Freund's adjuvant with 3 mg/ml of inactivated Mycobacterium tuberculosis (Difco Laboratories, Detroit, Mich.). Samples are obtained at sacrifice at 14, 23, and 41 days, in order to evaluate the extent of spinal cord mononuclear cell infiltration and the hematological changes.

### Chronic EAE Model (crEAE)

EAE was actively induced in inbred rats by subcutaneous inoculation into both footpads of syngenic whole myelin homogenate in Freund's adjuvant (100 mg/100 μl). After the onset of the clinical signs of EAE, the rats were stratified according to the severity of the clinical signs and randomly assigned on day 15 to one of the treatment groups. After deep anesthesia obtained by intraperitoneal injection of ketamine/xylazine mixture, all the surviving animals are sacrificed on day 60 and samples were obtained in order to evaluate the cardiotoxicity of the treatments, the hematological changes induced by the different schedules and the anti-MBP antibody titers.

### Monitoring of Immunosuppressive Activity

A method for monitoring pharmacodynamic drug action of a may be practiced prior to the administration of said drug to human subjects, however, myriad biochemical and metabolic pathways play a role in complex responses such as those collectively known as the immune system. Therefore, in addition to preclinical evaluation in animals, monitoring of patient responses to therapy is critical to the safe practice of the methods of the invention.

In preclinical evaluations, the immunosuppressive activity of an agent or treatment can be evaluated by assessment of the immune response of an animal, e.g. rabbits, when challenged with foreign antigens.

### Monitoring of Human Immune System Parameters

Immunosuppression may be due to the interruption multiple steps in immune activation such as inhibition of antigen presentation, cytokine production, and proliferation of lymphocytes. The concentration of peripheral blood leukocytes: lymphocyte, monocyte, basophils, neutrophils in circulation may decrease concomitantly or selectively and some populations may increase. For example, glucocorticosteriods produce immunosuppression via lymphocytopenia within 4 hours of administration. The peripheral lymphocyte count returns to normal within 24 to 48 hours. Corticosteroid-induced lymphocytopenia occurs as a result of redistribution of circulating lymphocytes into other lymphoid compartments (eg, spleen, lymph nodes, thoracic duct, and bone marrow). The recirculating lymphocyte pool, which accounts for approximately two thirds of the total lymphocyte pool, consists mainly of T lymphocytes (T cells) that migrate to and from the intravascular compartment and lymphoid tissue. Non-recirculating lymphocytes, which include some T cells and many B lymphocytes (B cells), live out their life span in the vascular compartment. Leukopenia can be functionally defined as WBC<4000 cells/mm³.

Monocytes/macrophages (promonocytes in the bone marrow, circulating monocytes, tissue macrophages) play a major role in the induction and regulation of immune reactivity. Macrophages are intricately involved in the presentation of antigens to lymphocytes and in the subsequent removal of immune complexes. Therefore, pharmacologic manipulation of these cells may directly and indirectly impair the immune response in general. Depletion of monocytes, characterized by cell counts decreasing from 300 to 400 cells/mm³ to <50 cells/mm³ is termed monocytopenia and inhibits inflammation by blocking responses to chemotactic factors and macrophage activation factor, phagocytosis, pyrogen production, and secretion of collagenase, elastase, and plasminogen activator.

Myelosuppression or neutrocytopenia is frequently associated with the administration of cytotoxic chemotherapeutic agents particularly those used to treat various malignancies. In addition, to assess a patient's hematologic status and ability to tolerate myelosuppressive chemotherapy, a complete blood count and platelet (thrombocyte) count should be obtained before chemotherapy is administered. Regular monitoring of hematocrit value and platelet count is recommended. Neutropenia, or low neutrophil count, is an absolute neutrophil count (ANC)<1500 cells/mm³ while severe neutropenia is defined as ANC<500/mm³. The duration of neutropenia is also a substantial parameter to monitor. Supportive therapy for myelosuppresion, such as the administration of recombinant granulocyte colony stimulating factor (e.g. NEUPOGEN®) therapy can be used to avoid or correct low neutrophil counts and can be discontinued if the ANC surpasses 10,000/mm³. Thrombocytopenia is defined as <100,000 cells/mm³.

In some cases an increase in the neutrophil count by 2000 to 5000 cells/mm³ (neutophilia) can also lead to immunosuppression by causing an accelerated release of neutrophils from the bone marrow into the circulation and a reduction in the migration of neutrophils out of the circulation. Also inhibition of the ability of neutrophils to adhere to vessel walls, which is an essential step in the migration of cells from the circulation into the tissue. The net effect is a reduced number of neutrophils available to accumulate at the inflammatory site.

Eosinophilia, manifested by a decrease in the eosinophil count to <25 cells/mm³, affects chemotaxis and may result from the inhibition of responses to chemotactic factors. Granulocytopenia is defined as <2000 cells/mm³.

Indirect effects, such as on the production of prostaglandin, may also be immunosuppressive and anti-inflammatory.

While the benefits of immunosuppression go hand in hand reduction of inflammatory sequelae, the adverse effects require careful monitoring of drug therapy. Other adverse effects associated generally with anthracyclines cumulative cardiac toxicity. Functional cardiac changes including decreases in left ventricular ejection fraction (LVEF) and irreversible congestive heart failure can occur with the use of mitoxantrone.

Secondly, the complications of immunodeficiency include the possibility of opportunistic infection and elevated incidence of certain malignancies.

Mitoxantrone use has been associated with an increased incidence of acute myelogenous leukemia in multiple sclerosis patients taking it. Secondary acute myelogenous leukemia (AML) has been reported in multiple sclerosis and cancer patients treated with mitoxantrone. In a cohort of mitoxantrone treated MS patients followed for varying periods of time, an elevated leukemia risk of 0.25% ( 2/802) has been observed. Postmarketing cases of secondary AML have also been reported. The use of mitoxantrone concomitantly with other cytotoxic agents and radiotherapy, increased the cumulative risk of developing treatment-related AML.

### Measurement of IL6

IL6 can be detected in bioassays employing IL6 responsive cell lines (7TD1; B9; CESS, KPMM2, KT-3; M1, MH60-BSF-2, MO7E; Mono Mac 6; NFS-60; PIL-6; SKW6-C14; T1165; XG-1). IL6 can be assayed also by its activity as a hybridoma growth factor due to the fact that most hybridomas are a result of the fusion of a myelogenous cell (myeloma) and a B-lymphocytes. Sensitive immunoassays and colorimetric tests are also available. An alternative detection method is RT-PCR quantitation of cytokines. Conventional solid or liquid phase competitive binding assays, e.g. ELISA assay, are available such as one using the receptor-associated gp130 protein (such reagents are available from e.g. R&D Systems).

For detection of IL6 bound to CNTO328, the anti-ID (anti-variable region antibodies disclosed in applicants copending applications U.S. Ser. No. 10/280,716 may be used to detect in any standard immunoassay format such as an ELISA-type assay.

### Diseases Amenable to Treatment by the Method of the Invention

The deregulated expression of IL6 is probably one of the major factors involved in the pathogenesis of a number of diseases. IL-6 is able to promote tumor growth by upregulating antiapoptotic and angiogenic proteins in tumor cells. The excessive overproduction of IL6 (and other B-cell differentiation factors) has been observed in various specific pathological conditions such as rheumatoid arthritis, multiple myeloma, Lennert syndrome (histiocytic lymphoma), Castleman's disease (lymphadenopathy with massive infiltration of plasma cells, hyper gamma-globulinemia, anemia, and enhanced concentrations of acute phase proteins), cardiac myxomas and liver cirrhosis. Constitutive synthesis of IL6 by glioblastomas and the secretion of IL6 into the cerebrospinal fluid has been observed.

With respect to immune mediated inflammatory diseases (IMIDs), IL6 is implicated in the pathogenesis of chronic polyarthritis (together with IL1 and IL8) since excessive concentrations of IL6 are found in the synovial fluid. In inflammatory intestinal diseases elevated plasma levels of IL6 may be an indicator of disease status. In patients with mesangial proliferative glomerulonephritis elevated urine levels of IL6 are also an indicator of disease status. IL6 may play a role in the immune mediated pathogenesis of diabetes mellitus of both type I and type II.

Accordingly, the present invention also provides a method for modulating or treating at least one IL-6 related disease, in a cell, tissue, organ, animal, or patient, as known in the art or as described herein, using at least one IL-6 antibody of the present invention, e.g., administering or contacting the cell, tissue, organ, animal, or patient with a therapeutic effective amount of IL-6 antibody in conjunction with administration of a immunosuppressive anthracenedione. The present invention also provides a method for modulating or treating at least one IL-6 related disease, in a cell, tissue, organ, animal, or patient including, but not limited to, at least one of obesity, an immune related disease, a cardiovascular disease, an infectious disease, a malignant disease or a neurologic disease.

The present invention also provides a method for modulating or treating at least one IL-6 related immune related disease, in a cell, tissue, organ, animal, or patient including, but not limited to, at least one of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, osteolysis, aseptic loosening of orthopedic implants, inflammatory bowel disease, ulcerative colitis, systemic lupus erythematosus, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, diabetes, nephrosis, atopic diseases, hypersensitity reactions, allergic rhinitis, hay fever, perennial rhinitis, conjunctivitis, endometriosis, asthma, urticaria, systemic anaphalaxis, dermatitis, pernicious anemia, hemolytic disesease, thrombocytopenia, graft rejection of any organ or tissue, kidney transplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, antibody-meditated cytotoxicity, type III hypersensitivity reactions, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetic retinopathy, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, OKT3 therapy, anti-cd3 therapy, cytokine therapy, chemotherapy, radiation therapy (e.g., including but not limited to, asthenia, anemia, cachexia, and the like), chronic salicylate intoxication, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

The present invention also provides a method for modulating or treating at least one cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, restenosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), post perfusion syndrome, cardiopulmonary bypass inflammation response, chaotic or multifocal atrial tachycardia, regular narrow QRS tachycardia, specific arrythmias, ventricular fibrillation, His bundle arrythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occlusion of the abdominal aorta and its branches, peripheral vascular disorders, occlusive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphederma, lipedema, unstable angina, reperfusion injury, post pump syndrome, ischemia-reperfusion injury, and the like. Such a method can optionally comprise administering an effective amount of a composition or pharmaceutical composition comprising at least one anti-IL-6 antibody to a cell, tissue, organ, animal or patient in need of such modulation, treatment or therapy.

The present invention also provides a method for modulating or treating at least one IL-6 related infectious disease in a cell, tissue, organ, animal or patient, including, but not limited to, at least one of: acute or chronic bacterial infection, acute and chronic parasitic or infectious processes, including bacterial, viral and fungal infections, HIV infection/HIV neuropathy, meningitis, hepatitis (e.g., A, B or C, or the like), septic arthritis, peritonitis, pneumonia, epiglottitis, e. coli 0157:h7, hemolytic uremic syndrome/thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, lyme disease, influenza a, epstein-barr virus, viral-associated hemaphagocytic syndrome, viral encephalitis/aseptic meningitis, and the like.

The present invention also provides a method for modulating or treating at least one IL-6 related malignant disease in a cell, tissue, organ, animal or patient, including, but not limited to, at least one of: leukemia, acute leukemia, acute lymphoblastic leukemia (ALL), acute lymphocytic leukemia, B-cell, T-cell or FAB ALL, acute myeloid leukemia (AML), acute myelogenous leukemia, chromic myelocytic leukemia (CML), chronic lymphocytic leukemia (CLL), hairy cell leukemia, myelodyplastic syndrome (MDS), a lymphoma, Hodgkin's disease, a malignant lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

The present invention also provides a method for modulating or treating at least one IL-6 related neurologic disease in a cell, tissue, organ, animal or patient, including, but not limited to, at least one of: neurodegenerative diseases, multiple sclerosis, migraine headache, AIDS dementia complex, demyelinating diseases, such as multiple sclerosis and acute transverse myelitis; extrapyramidal and cerebellar disorders, such as lesions of the corticospinal system; disorders of the basal ganglia; hyperkinetic movement disorders, such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi-system disorder); demyelinating core disorders, such as multiple sclerosis, acute transverse myelitis; and disorders of the motor unit’ such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; Dementia pugilistica; neurotraumatic injury (e.g., spinal cord injury, brain injury, concussion, repetitive concussion); pain; inflammatory pain; autism; depression; stroke; cognitive disorders; epilepsy; and the like. Such a method can optionally comprise administering an effective amount of a composition or pharmaceutical composition comprising at least one TNF antibody or specified portion or variant to a cell, tissue, organ, animal or patient in need of such modulation, treatment or therapy. See, e.g., the Merck Manual, 16^{th} Edition, Merck & Company, Rahway, N.J. (1992).

### Methods of Administration

The method of the present invention comprises administering an effective amount of a composition or pharmaceutical composition comprising at least one anti-IL-6 antibody to a cell, tissue, organ, animal or patient in need of such modulation, treatment or therapy in conjunction with treatment comprising administration of a immunosuppressive anthracenedione. The method of the invention comprises treating such diseases or disorders, wherein the administering of said at least one IL-6 antagonist is indicated. The method of the invention further comprises the co-administration with the IL6 antagonist, before, concurrently, and/or after, at least one immunosuppressive anthracenedione. In a specific embodiment, the IL6 antagonist is an antibody which prevents or inhibits the biological functions of IL6, such as a neutralizing IL6 antibody or an anti-IL6R antibody, and the immunosuppressive anthracenedione is selected from the group consisting of mitoxantrone, ametantrone, and pixantrone.

When mitoxantrone is used to treat acute myeloid leukemia; includes myelogenous, promyelocytic, monocytic, and erythroid acute leukemias: the dosage for induction is 12 mg/m(2) IV daily on days 1-3, in combination with cytarabine 100 mg/m(2) daily as continuous IV infusion on days 1-7. If incomplete response to the first induction, a second induction dose, 12 mg/m(2) IV daily for 2 days in combination with cytarabine 100 mg/m(2) daily as continuous IV infusion on days 1-5 may be given. A consolidation dose of 12 mg/m(2) IV daily on days 1 and 2, in combination with cytarabine 100 mg/m(2) daily as continuous IV infusion on days 1-5 is used; the first course is usually started 6 wk after final induction dose and the second, 4 weeks after the first.

Mitoxantrone injection is indicated for reducing neurologic disability and/or the frequency of clinical relapses associated with secondary progressive, progressive relapsing, or worsening relapsing-remitting multiple sclerosis. When used to treat multiple sclerosis, secondary progressive, progressive relapsing, or worsening relapsing-remitting; to reduce neurologic disability and/or frequency of clinical relapses: 12 mg/m(2) is given IV every 3 months. Mitoxantrone should not be administered to patients who have received a cumulative dose of 140 mg/m² or greater or patients with neutrophil counts less than 1,500 cells/mm³.

When used to treat patients diagnosed with prostate cancer, mitoxantrone is used in combination with corticosteroids, for pain related to advanced hormone-refractory prostate cancer: 12-14 mg/m(2) IV every 21 days, in combination with corticosteroids.

Typically, treatment of pathologic conditions is effected by administering an effective amount or dosage of an anti-IL-6 antibody composition that total, on average, a range from at least about 0.01 to 500 milligrams of at least one anti-IL-6 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 microgm/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

For parenteral administration, the antibody or the immunosuppressive anthracenedione can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Liposomal formulations of mitoxantrone (WO0232400A 1) and pixantrone have been described in e.g. EP1221940 B1 “Liposome formulation of 6,9-bis-(2-aminoethyl)-amino|benzog|isoquinoline-5,10-dione dimaleate”. The use of these or similar formulations to deliver the immunosuppressant antracenedione are within the scope of the method of the invention.

Suitable pharmaceutical carriers are described in the most recent edition of Remington's Pharmaceutical Sciences, A. Osol, a standard reference text in this field.

### Administration

Many known and developed modes can be used according to the present invention for administering pharmaceutically effective amounts of the IL6 antagonist and immunosuppressive anthracenedione according to the present invention. While parenteral administration is a typical, other modes of administration can be used according to the present invention with suitable results. Composition of the present invention can be delivered in a carrier, as a solution, emulsion, colloid, or suspension, or as a dry powder, using any of a variety of devices and methods suitable for administration by inhalation or other modes described here within or known in the art.

Alternative routes of administration include subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means.

Appropriate formulations comprising the active, IL6 antagonist and/or immunosuppressant anthracenedione, and one or more pharmaceutically approved excipients or diluents are encompassed by the present invention for use in the method of treatment of the invention.

### EXAMPLE 1

### In Vitro Activity of Anti-IL6 Antibody in Combination with Mitoxantrone

DU145 cells were established from a metastatic central nervous system lesion of androgen-independent prostate carcinoma (Stone, K. R. et al. 1978 Int J Cancer 21: 274-81). DU145 cells and the similarly androgen-independent prostate cell line PC-3 have been previously shown to exhibit autocrine secretion of IL-6 which is a resistance factor for etoposide and cisplatin-mediated cytotoxicity (Borsellino, N. et al. 1995 Cancer Res 55: 4633-9). Further, blocking of the common IL6 and concostatin M receptor (gp130) signaling was shown to inhibit PC-3 prostate tumor cells growth and sensitize the cells to etoposide and cisplatin cytotoxicity. Thus, study of direct interactions of chemotherapy agents and IL6 modulators can be studied on prostate tumor cells in vitro.

DU145 cells were plated at 500 cells/well in a 96 well plate and allowed to adhere for several hours. Treatments were added and plates were incubated at 37° C. for either 24, 48, or 72 hours. Concentrations of Mitoxantrone tested were 10, 1, 0.1, 0.03, 0.01, 0.003, 0.001, and 0 μM. CNTO 328 was tested at 20 μg/mL and IL-6 was tested at both 10 and 50 ng/mL. Plates were analyzed for ATP production using the ATPlite assay from Perkin Elmer. For graphing purposes, the media control (0 μM Mitoxantrone) point was given a value of 0.0001 μM. Results were expressed as % 0 μM Mitoxantrone treatment for each treatment group.

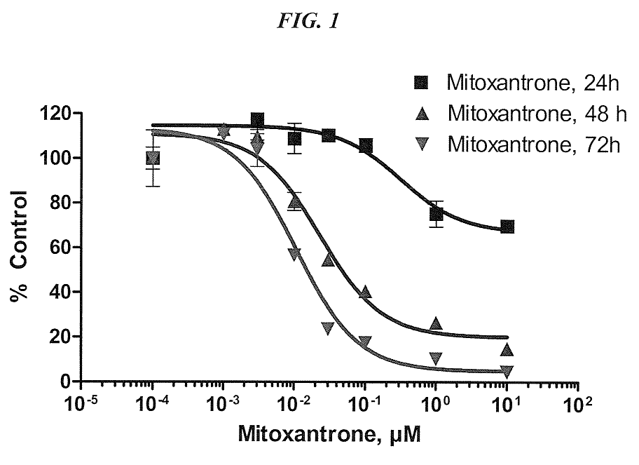

A timecourse of cell killing by mitoxantrone was performed which showed minimal cell death at 24 hours mitoxantrone and the greatest reduction in cell survival at 48 and 72 hours (FIG. 1).

The effect of anti-IL6 (CNTO 328) or IL-6 on the cytotoxic activity of mitoxantrone was tested. Incubation of cells with mitoxantrone in the presence of CNTO 328 (20 micrograms/mL) did not result in greater cell killing compared to mitoxantrone alone at the 48 hour or 72 hour timepoints.

TABLE 2
EC50 values for killing by Mitoxantrone (in nM)

| | | Mitoxantrone + | Mitoxantrone + | Mitoxantrone + |
|---|---|---|---|---|
| | Mito- | CNTO | IL- | IL- |
| | xantrone | 328 | 6 (50 nM) | 6 (10 nM) |
| 48 Hours | 23.03 | 22.92 | 23.17 | 24.34 |
| 72 Hours | 10.76 | 8.79 | 6.75 | 6.49 |

As CNTO328 is ineffective at reducing proliferation of the DU145 cells (not shown), these results indicate that CNTO328 does not significantly alter the sensitivity of the cells to mitoxantrone. Conversely, addition of exogenous IL-6 did not increase resistance to the effect of mitoxantrone at either timepoint.

### EXAMPLE 2

### In Vivo Activity of Anti-IL6 Antibody in Combination with Mitoxantrone

Nude mice were implanted subcutaneously with 1 mm3 fragments of DU-145 human prostate xenograft tissue. After 21 days (Day 1 of study), the animals were divided into four groups, n=8, each having a mean tumor volumes of 121-122 mm³. Dosing was initiated on Day 1 as follows: Group 1 (control) PBS i.p. biweekly; Group 2 CNTO328 10 mg/Kg i.p. biweekly; Group 3 Mitoxantrone 0.75 mg/Kg i.v. once per D for 5 D; Group 4 both CNTO328 and Mitoxantrone as in Groups 2 and 3. When dosed on the same day, CNTO 328 was given immediately prior to mitoxantrone.

The study endpoint was the time taken for tumors to reach a volume of 1000 mm3 at which time each animal was euthanized. Days in Progress=56. Calculation using the TTE=time to endpoint are: T−C=difference between median TTE (days) of treated versus control group, TGD=tumor growth delay. % TGD=[(T-C)/C]×100. The Logrank test was used to analyze the significance of the differences between the TTE values of treated and control groups. Two-tailed statistical analyses were conducted at significance level P-0.05. Statistical Significance: ns=not significant, **=P<0.01 compared to group indicated.

| | | Median | | | Statistical | |
|---|---|---|---|---|---|---|
| | | TTE | | % | Significance | |
| Group | Agent(s) | (Days) | T-C | TGD | Vs G1 | Vs G3 |
| 1 | PBS | 21.5 | — | — | — | — |
| 2 | CNTO328 | 16.9 | −4.6 | −21% | ns | — |
| 3 | Mitoxantrone | 27.7 | 6.2 | 29% | ns | — |
| 4 | CNTO328 + | 43.1 | 21.6 | 100% | P < 0.01 | P < 0.01 |
| | Mitoxantrone | | | | | |

The results of the experiment were that CNTO 328 (Group 2) or mitoxantrone monotherapy (Group 3) caused no significant tumor growth delay vs. control (Group 1). The combination of CNTO 328 and mitoxantrone produced a median TTE of 43.1 days representing a 100% tumor growth delay. No treatment-related deaths were reported, and the mean body weight nadir in the mitoxantrone group (−5%, day 10) was greater than in the combination group (−0.8%, day 10). These results demonstrate a synergistic anti-tumor effect of CNTO 328 in combination with mitoxantrone over either agent alone in a model of human prostate cancer in so far as the % TGD of each agent alone was less than the % TGD in animals administered CNTO328 and mitoxantrone and the sum of the % TGD for each agent is less than % TGD in the group treated with the combination.

### EXAMPLE 3

### Treatment of HRPC Patients with Antibody to IL6 and Mitoxantrone

Mitoxantrone in combination with prednisone (M/P) has long been the reference cytotoxic treatment for metastatic HRPC, based on clinical trial data showing significant palliative benefits but despite a lack of survival benefit. Recent results of major Phase 3 trials comparing treatment regimens which include docetaxel, the TAX 327 study (Tannock et al., 2004 N Engl J Med. 351(15):1502-1512), and the SWOG 9916 trial (Petrylak et al., 2004 N Engl J Med. 351(15):1513-1520), consistent improvement of survival in patients treated with docetaxel every 3 weeks of approximately 2-2.5 months over the reference M/P regimen. Therefore, in addition to further improvement in survival of HRPC patients, there is a need for to provide a treatment option for HRPC patients with metastatic disease who have either relapsed or are refractory to prior docetaxel treatment.

The first study of anti-IL6 antibody (CNTO328) treatment combined with mitoxantrone in human subjects is a 2-part, open-label, multicenter, Phase 2 study of the safety and efficacy of the combination versus mitoxantrone in subjects with metastatic HRPC who have received one prior docetaxel-based chemotherapy regimen.

Eligible subjects must be age 18 years, have radiologically documented metastatic disease, received at least 6 weeks of docetaxel for HRPC, and have disease progression during or within 3 months after cessation of docetaxel-based therapy. Subjects must have normal cardiac function, as evidenced by a left ventricular ejection fraction (LVEF)³ 50%. Approximately 143 subjects will be enrolled in the study (9 in Part 1, and 134 in Part 2, randomized to 2 arms). All evaluable subjects will be included in the analyses. The safety and efficacy of the combination of CNTO 328 plus mitoxantrone will be evaluated in Part 1, and the study will proceed to the randomized portion (Part 2), provided the safety profile of the combination is comparable to historical mitoxantrone data.

Part 1 of the study is single arm and open label. Subjects will receive mitoxantrone, prednisone, and CNTO 328. Mitoxantrone will be administered at a dose of 12 mg/m² IV as a 30 minute infusion on Day 1 of each 3-week cycle, until disease progression or unacceptable toxicity or up to 10 cycles (a maximum cumulative dose of approximately 120 mg/m²). CNTO 328 will be administered at 6 mg/kg IV as a 2 hour infusion, starting Day 1 of Cycle 1 to continue every 2 weeks until disease progression or unacceptable toxicity or up to a maximum of 1 year.

Part 2 of the study is the randomized portion, consisting of 2-arms, randomized in 1:1 ratio. The experimental arm (Arm A) will consist of mitoxantrone (M), prednisone (P), and CNTO 328. Mitoxantrone will be administered at a dose of 12 mg/m² IV as a 30 minute infusion on Day 1 of each 3-week cycle until disease progression or unacceptable toxicity or up to 10 cycles (a maximum cumulative dose of approximately 120 mg/m²). CNTO 328 will be administered at a dose of 6 mg/kg IV as a 2 hour infusion, starting Day 1 of Cycle 1 to continue every 2 weeks until disease progression or unacceptable toxicity or up to a maximum of 1 year. The control arm (Arm B) will consist of treatment with M/P. Mitoxantrone will be administered at a dose of 12 mg/m2 IV as a 30 minute infusion on Day 1 of each 3-week cycle, until disease progression or unacceptable toxicity or up to 10 cycles (a maximum cumulative dose of approximately 120 mg/m²).

This study is designed to evaluate the hypothesis that treatment with the combination of CNTO 328 plus mitoxantrone is superior to treatment with mitoxantrone in prolongation of the progression-free survival of subjects with HRPC. The primary analysis will include all randomized subjects. PFS for the 2 treatment arms will be compared using log rank test at 2-sided a level of 0.05. The major secondary endpoints (in order of importance) to be summarized are: 1) time to clinical deterioration 2) palliative response 3) PSA response and 4) overall survival.

Disease progression, during or within 6 months of cessation of prior docetaxel-based therapy, is based on one of the following
a. Serum PSA progression, defined as a rise in at least 2 consecutive serum PSA values, each obtained at least 1 week apart, or
b. Radiological disease progression: if disease progression is shown by bone scan only, then disease progression is defined by the appearance of two or more new bone lesions.
In addition, the subjects will have had orchiectomy or have testosterone <50 ng/dL by means of pharmacological/chemical castration and therefore are diagnosed with hormone refractive adenocarcinoma of the prostate.

The duration of treatment will be a maximum of 12 months for CNTO 328 or approximately 7 months for mitoxantrone, based on the maximum cumulative dose. A radiologist at the study site will evaluate tumor response to treatment. Tumor response will be assessed using Response Evaluation Criteria in Solid Tumors (RECIST) criteria (Therasse et al, 2000; see Appendix A. PSA will be evaluated on Day 1 of every cycle (ie, every 3 weeks).

Other parameters of immune competency and immune function, as well as cardiac function, serum markers such as PSA, testosterone, and standard blood chemistry will be monitored on a proscribed schedule. Pain and the need for pain relief will be evaluated by predetermined methods.

### Surrogate Markers

Since IL-6 is associated with disease activity and CRP is a surrogate marker of IL-6 activity, sustained suppression of CRP by neutralization of IL6 by CNTO 328 may be assumed necessary to achieve biological activity. The relationship between IL-6 and CRP in patients with benign and malignant prostate disease was examined by McArdle (McArdle et al. 2004 Br J Cancer 91(10):1755-1757). Although they found no significant differences between the concentrations of IL-6 and CRP in the patients with benign disease compared with prostate cancer patients, in the cancer patients there was a significant increase in both IL-6 and CRP concentration with increasing tumor grade. The median serum CRP value for the 86 subjects with prostate cancer was 1.8 mg/L. Therefore, the proposed dose and schedule for the current study of 6 mg/kg CNTO 328 administered every 2 weeks is likely to achieve sustained suppression of CRP in subjects with metastatic HRPC.

### General Statistical Methods

For continuous parameters, number of observations, means, standard deviations, medians, and ranges will be used. For discrete parameters, frequency will be summarized. For time-to-event parameters, Kaplan-Meier estimates, hazard ratio and its 95% confidence interval will be provided.

The secondary efficacy analyses includes time-to-event analyses performed on the observed distributions of time-to-event endpoints. These observed distributions of time-to-event endpoints are compared between regimens using the log-rank test. Wilcoxon test are used as a secondary comparison between treatment arms. Additional supporting analyses include Kaplan-Meier estimation (Kaplan and Meier, 1958 J Am Stat Assoc. 53:457-481) by regimen. Other time-to-event analyses are performed as deemed necessary.

Having exemplified the invention, the invention is further defined by the appended claims.

What is claimed:

1. A method of treating a subject suffering from prostate cancer whereby said patient is in need of such treatment, which comprises co-administering an immunosuppressive 9,10-anthracenedione in combination with a neutralizing IL-6 antibody.

2. The method according to claim 1, in which the IL-6 antibody is cCLB8 or comprises cCLB8 or a fragment thereof.

3. The method according to claim 2 in which the antibody is a monoclonal antibody.

4. The method according to claim 2, in which the antibody or fragment binds to IL6.

5. The method according to claims 3 or 4, in which the antibody fragment is an Fab, Fab′, or F(ab′)2 fragment or derivative thereof.

6. The method according to claim 3, in which the monoclonal antibody competes with monoclonal antibody cCLB8 for binding to human IL6.

7. The method according to claim 3, in which the monoclonal antibody is administered intravenously

8. The method according to claim 3, in which the monoclonal antibody is administered in the amount of from 0.01 mg/kg to 12.0 mg/kg body weight.

9. The method according to claim 3, in which the monoclonal antibody is administered in a bolus dose followed by an infusion of said antibody.

10. The method according to claim 1, in which the subject is a human patient diagnosed with hormone refractive prostate cancer.

11. The method according to claim 1 in which the anthracenedione is mitoxantrone or pixantrone.

12. The method according to claim 1 in anthracenedione suppresses myelin degradation in a rat EAE model.

13. The method according to claim 1, in which the subject is diagnosed with prostate cancer and exhibits additional symptoms selected from the group consisting of hypercalcemia, metastatic lesions, or cachexia.

14. The method according to claim 1 in which the anti-IL6 antagonist is administered sequentially, serially, or concurrently with the immunosuppressive anthracenedione.

15. A method for inhibiting tumor growth in a mammal in need thereof comprising administering to the mammal in conjunction with an immunosuppressive anthracenedione, a monoclonal antibody or fragment thereof which prevents IL6 activation of signaling through membrane bound receptors in an amount effective to inhibit the growth of said tumor.

16. A method for treating a patient diagnosed with prostate cancer and having extracapsular extensive disease, comprising administering to the mammal in conjunction with an immunosuppressive anthracenedione, a monoclonal antibody or fragment thereof which prevents IL6 activation of signaling through membrane bound receptors in an amount effective to prevent the growth or reduce pain in said mammal.

17. A method of treating an IL-6 related disorder or condition, in a mammal in need of such treatment, which comprises co-administering an immunosuppressive anthracenedione in combination with an IL-6 antagonist.
